(19) United States
(12) Patent Application Publication
(10) Pub. No.: US 2005/0095293 A1
(43) Pub. Date: May 5, 2005

(54) Administration form for the oral application of poorly soluble drugs

(75) Inventors: Ulrich Brauns, Biberach (DE); Rolf-Stefan Brickl, Warthausen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH Co. KG, Ingelheim (DE)

(21) Appl. No.: 10/934,140

(22) Filed: Sep. 3, 2004

(30) Foreign Application Priority Data
Mar. 7, 2002 (DE) DE 102 09 982.0

(51) Int. Cl. A61K 31/275; A61K 9/26

(52) U.S. Cl. 424/469

(57) ABSTRACT

The invention relates to a new formulation for oral administration of active substances with pH-dependent solubility characteristics and the pharmacologically acceptable salts thereof, which improves the bioavailability of the active substance.

### FIELD OF THE INVENTION

The invention relates to a formulation for the oral administration of basic active substances with pH-dependent solubility characteristics, and the salts thereof.

### DESCRIPTION OF THE FIGURES

FIG. 1 shows the schematic structure of a pharmaceutical composition according to the invention in the form of a sectional view of a pellet.

FIG. 2 graphically shows the bioavailability of the formulations according to the invention compared with the conventional tablet with and without the addition of pantoprazole.

FIG. 3 shows the in vitro releases of an Example C5 according to the invention compared with the reference formulation C2 with a similar active substance content in 0.005 mol citrate buffer pH 5.0.

### DESCRIPTION OF THE INVENTION

The term “active substance” for the purposes of this invention refers to any pharmacologically effective compound which (as such or in the form of the pharmaceutically acceptable salts thereof) is a weak base and in the range from pH 1 to pH 7.5 exhibits pH-dependent solubility characteristics (with greater solubility under acidic conditions and less solubility under basic conditions). In these active substances, in fact, the bioavailability may be dependent on the pH in the gastrointestinal tract when administered orally. Preferably, active substances in the sense of this invention have a relatively high saturation solubility in aqueous solutions at low pH levels as a result of single or multiple protonation, whereas at pH values above 5 the neutral compounds are virtually insoluble according to the definition in the European Pharmacopoeia (saturation solubility less than 100 μg/ml).

The oral formulation according to the invention may contain as active substance for example ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate (WO 98/37075), BIBU 104 (Lefradafiban; (3S,5S)-5-[[4′-(N-methoxycarbonylamidino)4-biphenylyl]-oxymethyl]-3-[(methoxycarbonyl)methyl]-2-pyrrolidinone; EP 0 483 667), (R)-2-[4-(N-phenylcarbonylamidino)-phenylaminomethyl]-1-methyl-5-[1-(n-propyloxycarbonyl-methylamino)-1-(pyrrolidinocarbonyl)-ethyl]-benzimidazole (WO 01/47896), telmisartan, DTTX 30 SE, BIBV 308 SE (terbogrel), bromhexine, BIIL 284 (amelubant; 4-((3-((4-(1-(4-hydroxyphenyl)-1-methylethyl)phenoxy)methyl)benzyl)oxy)benzenecarboximidamid-N-ethylcarboxylate; WO 96/02497), flibanserin (1-[2-(4-(3-trifluoromethyl-phenyl)piperazin-1-yl)ethyl]-2,3-dihydro-1H-benzimidazol-2-one; EP-A-526434;), 4-(4-(2-pyrrolylcarbonyl)-1-piperazinyl)-3-trifluoromethyl-benzoylguanidine (WO 00/17176), pimobendane or one of the pharmacologically acceptable salts of these compounds (such as the hydrochlorides, hydrobromides, mesylates, sulphates and phosphates).

The solubility of a compound may be determined by dispersing an excess of the compound at ambient temperature in the medium in question and shaking it vigorously for a defined length of time (approx. 1 to 24 h) until equilibrium is achieved. After filtration the pH is determined in the clear filtrate and the concentration of the dissolved substance is determined by spectral photometry or some other suitable analytical process.

The pH-dependent solubility characteristics of the active substance may mean that, depending on the dose, when administered orally in solid preparations of conventional composition, the active substance is only totally dissolved in the patient's stomach if the liquid present in the stomach has a low enough pH. If the pH in the stomach is elevated (this may be the result of normal physiological variability, illness or co-medication with pharmaceutical compositions that raise the gastric pH), the active substance may not dissolve totally.

The effect of the dose of the active substance on its bioavailability can be quantitatively described by means of the concept of the (dimensionless) dose number (Do). The dose number is defined as:
Do=(Mo/Vo)/Cs,
where

Mo=dose (mg),

Vo=liquid volume present (ml) and

Cs=saturation solubility (mg/ml).

According to an assumption which is conventional nowadays the liquid volume in the stomach after taking a preparation is about 250 ml. (Löbenberg, R., Amidon, G. L.: Modern bioavailability, bioequivalence and biopharmaceutics classification system. New scientific approaches to international regulatory standards (Eur. J. Pharm. Biopharm. 50 (2000) 3-12).

At dosages which give a dose number of less than 1, no solubility problems occur. Only if the critical dose number of 1 is exceeded may there be significant reductions in solubility and hence a decreased bioavailability. As a rule the actual problem area only begins at doses which give a dose number significantly above 1, as at least some of the dissolved substance is constantly eliminated from the equilibrium by the absorption process.

The active substances contained in the oral formulation according to the invention have a value of less than 1 for the dose number, based on the solubility at pH<2 (i.e. a sufficiently acidic stomach) and a value significantly above 1 for the dose number based on the solubility at pH>5 (i.e. no or vanishingly low gastric acid), i.e. for the oral formulation according to the invention both the degree of pH-dependence of the solubility of the active substance and the size of the dose of active substance are of interest.

Raising the gastric pH in the case of active substances with solubility characteristics of this kind may then lead to a substantially reduced bioavailability of the active substance (even amounting to total malabsorption) which in some cases results in failure of the treatment. Generally increasing the dose in order to compensate for the reduced bioavailability in patients with a raised gastric pH is frequently undesirable because of the waste of active substance and the greater burden on the patient and the associated risk of side effects, for example, or even totally impossible, on the grounds of drug safety.

The aim of the invention is to provide a pharmaceutical composition for oral administration of active substances with pH-dependent solubility characteristics which guarantees largely pH-independent bioavailability of the active substance.

It has now been found that the use of pharmaceutically acceptable organic acids with a water solubility of more than 1 g/250 ml at 20° C., preferably more than 1 g/160 ml at 25° C., in solid oral formulations can ensure sufficient bioavailability of active substances with pH-dependent solubility characteristics, even in patients with a raised gastric pH.

Pharmaceutically suitable acids for the purposes of this invention are for example tartaric acid, fumaric acid, succinic acid, citric acid, malic acid, glutamic acid and aspartic acid including the hydrates and acid salts thereof. Particularly suitable for the purposes of this invention are tartaric acid, fumaric acid, succinic acid and citric acid, particularly tartaric acid, fumaric acid and succinic acid.

Numerous active substances display a more or less marked tendency to hydrolytic decomposition in the presence of acids and traces of water. In individual cases there may even be a direct chemical reaction between the active substance and organic acids, e.g. ester formation. When developing a product which remains stable when stored it is therefore advantageous to separate the organic acid spatially from the active substance in the formulation. Only after the administration of the formulation does the organic acid dissolve and produce an acidic microclimate in which the active substance can dissolve.

A further aim of the invention is to prevent the undesirable interactions between acid and active substance in spite of the use of an organic acid to improve the solubility.

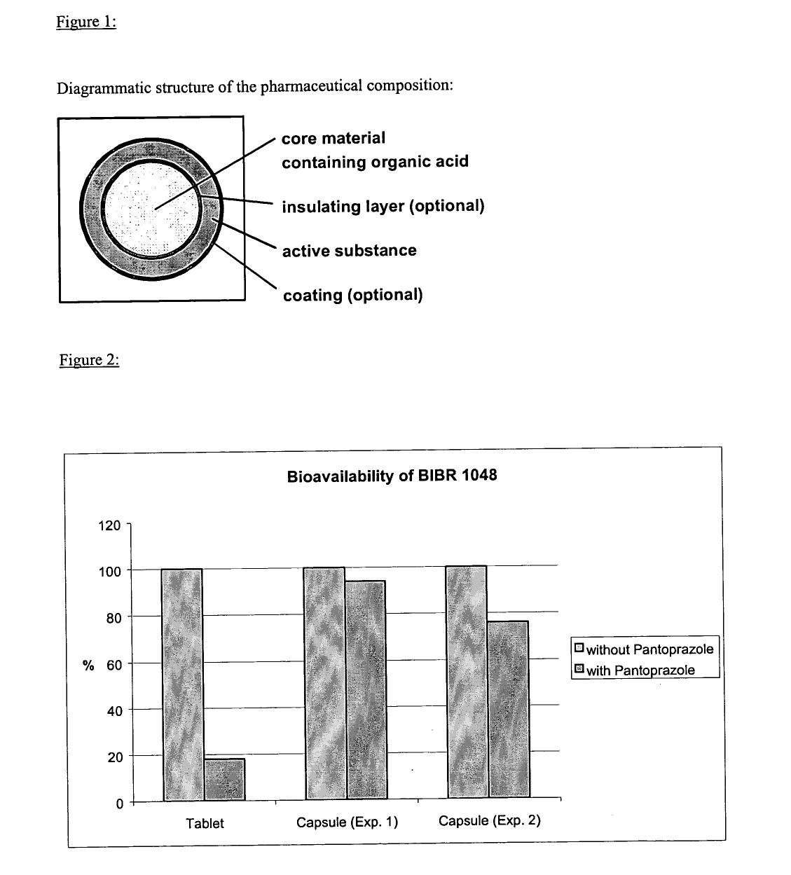

Multiparticulate formulations in which the individual particles have the structure shown in FIG. 1 are particularly suitable for the preferred spatial separation of active substance and organic acid.

FIG. 1 shows the diagrammatic structure of the pharmaceutical composition by means of a section through a pellet which is suitable for producing the pharmaceutical composition according to the invention. The roughly bead-shaped/spherical core portion of this pellet contains or consists of the pharmaceutically acceptable organic acid. This is optionally followed by a layer which separates the acid core from the layer containing the active substance, the so-called insulating layer. The insulating layer in turn, or the core material in the absence of an insulating layer, is surrounded by the active substance layer, which is also spherical, which may itself be surrounded by a coating to increase the abrasion resistance and shelf life of the pellets.

The core material used is a pharmaceutically acceptable organic acid with a water solubility of >1 g/250 ml at 20° C., such as e.g. tartaric acid, fumaric acid, succinic acid, citric acid, malic acid, glutamic acid and aspartic acid including the hydrates and acid salts thereof, to which a small amount of 1 to 10% by weight, preferably 3 to 6% by weight of a suitable binder is optionally added. The use of a binder may be necessary, for example, if the starting acids are produced by a pan build-up process. If the method used is extrusion or spheronisation, other technological adjuvants such as microcrystalline cellulose will be needed instead of binders. It is also possible to use pure (100%) acid as the starting material if it can be obtained in a sufficiently narrow range of particle sizes. The pharmaceutically acceptable organic acids used are preferably tartaric acid, fumaric acid, succinic acid or citric acid; tartaric acid is particularly preferred. As binder, it is possible to use gum arabic or a partially or totally synthetic polymer selected from among the hydroxypropylcelluloses, hydroxypropylmethylcelluloses, methylcelluloses, hydroxyethylcelluloses, carboxymethylcelluloses, polyvinylpyrrolidone, the copolymers of N-vinylpyrrolidone and vinyl acetate, or combinations of these polymers; gum arabic is preferred. The spherical core material preferably has an average diameter of 0.4-1.5 mm. The content of the pharmaceutically acceptable organic acid is usually between 30 and 100% in the core material.

To increase the durability of the finished product it is advantageous to coat the core material before the application of the active substance with an insulating layer based on a water-soluble, pharmaceutically acceptable polymer. Examples of such water-soluble polymers include for example gum arabic or a partially or totally synthetic polymer selected from among the hydroxypropylcelluloses, hydroxypropylmethylcelluloses, methylcelluloses, hydroxyethylcelluloses, carboxymethylcelluloses, polyvinylpyrrolidone, the copolymers of N-vinylpyrrolidone and vinyl acetate, or combinations of these polymers. Gum arabic or a hydroxypropylmethylcellulose is preferably used. If desired, the coating with the water-soluble, pharmaceutically acceptable polymer may be carried out with the addition of suitable plasticisers, separating agents and pigments, such as for example triethylcitrate, tributylcitrate, triacetin, polyethyleneglycols (plasticisers), talc, silicic acid (separating agents), titanium dioxide or iron oxide pigments (pigments).

The active substance layer contains the active substance as well as binders and optionally separating agents. Suitable binders include for example hydroxypropylcellulose, hydroxypropylmethylcellulose, methylcellulose, hydroxyethylcellulose, carboxymethylcellulose, polyvinylpyrrolidone, copolymers of N-vinylpyrrolidone and vinyl acetate or combinations of these polymers. Preferably, hydroxypropylcellulose or copolymers of N-vinylpyrrolidone and vinyl acetate are used. The addition of separating agents such as e.g. talc, magnesium stearate or silicic acid serves to prevent the particles from aggregating during the process. The preferred active substance content is not more than 60%, preferably not more than 50% of the pharmaceutical composition.

The optional outermost layer, which serves to reduce any increased abrasion during packing into capsules and/or to increase the shelf life, consists of pharmaceutically conventional film-forming agents, plasticisers and optionally pigments. Suitable film-forming agents include for example hydroxypropylcellulose, hydroxypropylmethylcellulose, methylcellulose, polymers and copolymers of acrylic and methacrylic acid and the esters thereof, or combinations of these polymers. Suitable plasticisers include inter alia triethylcitrate, tributylcitrate, triacetin or polyethyleneglycols. The pigments used may be e.g. titanium dioxide or iron oxide pigments. Preferably, the outer coating consists of hydroxypropylmethylcellulose and/or methylcellulose, optionally with the addition of polyethyleneglycols as plasticisers.

The pellets may be prepared by the method described hereinafter:

The acid-containing core material consists either of crystals of the particular organic acid used or, more advantageously, of roughly spherical particles of the desired size containing a large amount of organic acid, which can be produced by methods known and established in pharmaceutical technology. The core material may be produced, in particular, by pan methods, on pelleting plates or by extrusion/spheronisation. Then the core material thus obtained may be divided into fractions of the desired diameter by screening. Suitable core material has an average diameter of 0.4 to 1.5 mm, preferably 0.6 to 0.8 mm.

First, the insulating layer is applied to this acid-containing core material. This can be done by conventional methods, e.g. by applying an aqueous dispersion of the water-soluble, pharmaceutically acceptable polymer, optionally with the addition of plasticisers, separating agents and/or pigments, in a fluidised bed, in coating pans or in conventional film coating apparatus. If necessary the product can then be screened again.

Then the active substance is applied from a dispersion containing binder and optionally separating agent. The volatile dispersant is removed during or after the process by drying. Suitable binders in the dispersion may be for example hydroxypropylcellulose, hydroxypropylmethylcellulose, methylcellulose, hydroxyethylcellulose, carboxymethylcellulose, polyvinylpyrrolidone, copolymers of N-vinylpyrrolidone and vinyl acetate or combinations of these polymers. Preferably, hydroxypropylcellulose or copolymers of N-vinylpyrrolidone and vinyl acetate are used. Suitable separating agents include e.g. talc, magnesium stearate or silicic acid; preferably, talc is used. The dispersants may be for example water, ethanol, 2-propanol, acetone or mixtures of these solvents with one another. The application of active substance to the core material may be carried out by established methods known in pharmaceutical technology, e.g. in coating pans, conventional film coating apparatus or by the fluidised bed method. Then a further screening process may be carried out.

To reduce any increased abrasion during transfer into capsules or to increase the shelf life the system may finally be coated with a coating of a pharmaceutically conventional film forming agent, plasticiser and optionally pigment. This may be done by conventional methods as mentioned earlier in the description of the application of the insulating layer.

When core material with an average diameter of 0.4-1.5 mm is used, the process described above produces pellets containing active substance, which can then be packed into hard capsules, for example. To do this, a number of these units corresponding to the required dosage are packed into hard capsules in a standard capsule filling machine. Suitable hard capsules include, for example, hard gelatine capsules or hard capsules of hydroxypropylmethylcellulose (HPMC). The preferred active substance content of the pharmaceutical composition is not more than 60%, preferably not more than 50%.

Unless otherwise stated, percentages specified are always percent by weight. All the data on the active substance content relate to the active substance base unless otherwise stated.

As already mentioned, the pharmaceutical compositions according to the invention exhibit improved bioavailability of the active substance contained therein. For example, the pharmaceutical compositions according to Examples 1 and 2 were tested for the improvement to the bioavailability.

Results of the Clinical Trials

To do this, the formulation prepared according to Example 1 was clinically tested for its bioavailability on a total of 15 volunteers. In one branch of the treatment, the volunteers were given the composition by mouth (=orally) on an empty stomach without any pre-treatment. In another branch of the treatment the same volunteers were pre-treated, prior to the oral administration of the composition, with 40 mg of pantoprazole b.i.d. (=twice a day) for three days by mouth to increase the gastric pH; the treatment with pantoprazole was continued during the administration of the formulation according to the invention.

The degree of absorption was determined by measuring the quantity of active metabolite 3-[(2-{[4-(amino-imino-methyl)-phenylamino]-methyl}-1-methyl-1H-benzimidazole-5-carbonyl)-pyridin-2-yl-amino]-propionic acid excreted in the urine.

The relative bioavailability after pre-treatment with pantoprazole was 94% on average compared with administration without any pre-treatment.

Under comparable conditions of administration, the relative bioavailability (based on the area under the plasma concentration/time curve) of a tablet containing 50 mg of active substance, developed and produced according to the prior art and containing no water-soluble organic acid, after corresponding pre-treatment with pantoprazole, is 18%. The following list shows the precise composition of the tablet used:

TABLE 1

| Ingredient | mg/tablet |
|---|---|
| Core | |
| ethyl 3-[(2-{[4-(hexyloxycarbonyl- | 57.7 |
| amino-imino-methyl)-phenylamino]- | |
| methyl}-1-methyl-1H-benzimidazole-5- | |
| carbonyl)-pyridin-2-yl-amino]- | |
| propionate mesylate | |
| lactose monohydrate | 58.0 |
| microcrystalline cellulose | 48.3 |
| crospovidone | 3.4 |
| magnesium stearate | 2.6 |
| Film coating | |
| polyethyleneglycol 6000 | 0.56 |
| titanium dioxide | 0.80 |
| talc | 0.64 |
| hydroxypropylmethylcellulose | 1.92 |
| iron oxide yellow | 0.08 |
| Total | 174.0 |

The bioavailability was thus improved by about a factor of 5 by using the formulation according to the invention.

The formulation prepared according to Example 2 was also clinically tested for its bioavailability on a total of 15 volunteers. In one branch of the treatment, the volunteers were given the composition by mouth on an empty stomach without any pre-treatment. In another branch of the treatment the same volunteers were pre-treated, prior to the oral administration of the composition, with 40 mg of pantoprazole b.i.d. for three days by mouth to increase the gastric pH; the treatment with pantoprazole was continued during the administration of the formulation according to the invention.

The degree of absorption was determined by measuring the quantity of the active metabolite 3-[(2-{[4-(amino-imino-methyl)-phenylamino]-methyl}-1-methyl-1H-benzimidazole-5-carbonyl)-pyridin-2-yl-amino]-propionic acid excreted in the urine.

The relative bioavailability after pre-treatment with pantoprazole was 76% on average compared with administration without any pre-treatment.

Under comparable conditions of administration, the relative bioavailability (based on the area under the plasma concentration/time curve) of a tablet containing 50 mg of active substance, developed and produced according to the prior art and containing no water-soluble organic acid, after corresponding pre-treatment with pantoprazole, is 18%. The following list shows the precise composition of the tablet used:

TABLE 2

| Ingredient | mg/tablet |
|---|---|
| Core | |
| ethyl 3-[(2-{[4-(hexyloxycarbonyl- | 57.7 |
| amino-imino-methyl)-phenylamino]- | |
| methyl}-1-methyl-1H-benzimidazole-5- | |
| carbonyl)-pyridin-2-yl-amino]- | |
| propionate mesylate | |
| lactose monohydrate | 58.0 |
| microcrystalline cellulose | 48.3 |
| crospovidone | 3.4 |
| magnesium stearate | 2.6 |
| Film coating | |
| polyethyleneglycol 6000 | 0.56 |
| titanium dioxide | 0.80 |
| talc | 0.64 |
| hydroxypropylmethylcellulose | 1.92 |
| iron oxide yellow | 0.08 |
| Total | 174.0 |

The bioavailability of the active substance compared with conventional formulations was thus improved by about a factor of 4 by using the formulation according to the invention.

FIG. 2 graphically illustrates the bioavailability of the formulations according to the invention compared with the conventional tablet with and without the administration of pantoprazole.

The amount of active substance per capsule is preferably selected so that taking 1 to 2 capsules a day is sufficient to achieve the desired activity.

The preferred ratio of acid to active substance is about 1:1 to approx. 20:1. The theoretical lower limit at which the system can still function is 1 equivalent of acid per mol of active substance. The upper limit of approximately 20:1 (acid to active substance) is determined by the size of the formulation at the desired dosages (number of pellets per capsule).

In quality control, in vitro releases are measured by USP methods. The drug is released in a volume of 900 ml and the pH is selected so as to obtain “sink conditions”, i.e. the entire dose of active substance is soluble in these 900 ml. This in vitro method cannot be predictive of absorption in humans as a patient will generally take the drug with approx. 200 ml of liquid and in a non-acidic stomach the solubility is often only sufficient for a fraction of the dose. Non-acidic stomach occurs at a rate of about 25% of the population in older patients and is often also caused by co-medication with H2-blockers or proton pump inhibitors.

Therefore, within the scope of the invention, an empirical test method was developed which has a better correlation with the in vivo performance in humans, particularly in non-acidic stomach. In this procedure, a drug preparation which contains the maximum dose used in humans is released in a volume of 200 ml (this corresponds to the dose in humans) in buffer at a pH with reduced solubility of the active substance in the physiologically relevant range, i.e. between pH 1-7. As the absorbability can also be predicted with some accuracy using this method, even at non-acidic gastric pH levels, it is suitable for optimising drug preparations. In order to identify the most favourable formulation in each case from a number of possible recipes, the maximum release and/or the area under the curve (AUC) from time 0 to the end of the release may be used as relevant characteristics.

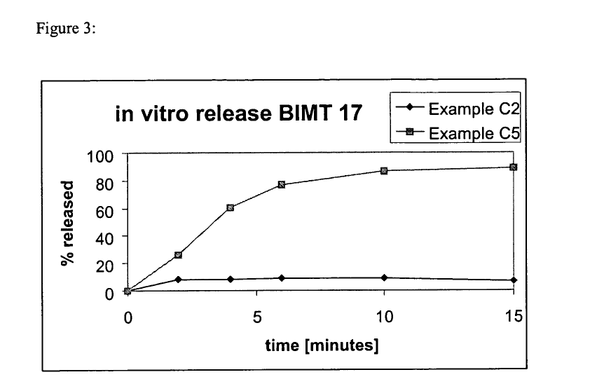

This is clear from the example of the comparison of the formulation examples C2 (reference without acid starter) and C5 (Example according to the invention with tartaric acid starter) (FIG. 3 and Table 3).

Table 3 shows the in vitro releases and the characteristics of the AUC and maximum release of an Examples C5 according to the invention (active substance: flibanserin; cf. Table 11) compared with the reference form with a similar content of active substance in 0.005 mol citrate buffer pH 5.0

TABLE 3

| time | | | | | | | AUC | max | |
|---|---|---|---|---|---|---|---|---|---|
| (min) | 0 | 2 | 4 | 6 | 10 | 15 | 0-15′ | release | pH* |
| Example | 0 | 8 | 8 | 9 | 9 | 7 | 115 | 9 | 4.90 |
| C2 | | | | | | | | | |
| Example | 0 | 26 | 61 | 77 | 87 | 89 | 1018 | 89 | 3.45 |
| C5 | | | | | | | | | |

*This value represents the pH at the end of the release.

Table 4 shows the in vitro releases and the characteristics of the AUC and maximum release of Examples C4 to C15 according to the invention (active substance: flibanserin; cf. Table 11) compared with the reference forms C1 to C3

TABLE 4

| | | | | | | | | max | |
|---|---|---|---|---|---|---|---|---|---|
| time (mins) | 0 | 2 | 4 | 6 | 10 | 15 | IC 0- | release | pH* |
| Example C1 | 0 | 8 | 9 | 9 | 9 | 8 | 119 | 9 | 4.90 |
| Example C2 | 0 | 8 | 8 | 9 | 9 | 7 | 115 | 9 | 4.90 |
| Example C3 | 0 | 5 | 7 | 8 | 8 | 6 | 97 | 8 | 4.29 |
| Example C4 | 0 | 65 | 91 | 95 | 97 | 101 | 1309 | 103 | 2.80 |
| Example C5 | 0 | 26 | 61 | 77 | 87 | 89 | 1018 | 89 | 3.45 |
| Example C6 | 0 | 11 | 19 | 29 | 45 | 50 | 476 | 50 | 3.95 |
| Example C7 | 0 | 13 | 34 | 53 | 80 | 99 | 884 | 107 | 3.00 |
| Example C8 | 0 | 5 | 11 | 22 | 55 | 100 | 598 | 100 | 4.00 |
| Example C10 | 0 | 51 | 79 | 86 | 94 | 97 | 1182 | 97 | 3.98 |
| Example C11 | 0 | 7 | 16 | 27 | 33 | 40 | 371 | 40 | 3.95 |
| Example C14 | 0 | 26 | 77 | 88 | 91 | 93 | 1113 | 93 | 3.35 |
| Example C15 | 0 | 16 | 36 | 37 | 36 | 37 | 471 | 37 | 3.95 |

*This value represents the pH at the end of the release.

Interpretation of the Results:

All the Examples according to the invention are clearly superior to the reference formulation. As the content of active substance increases the in vitro release declines somewhat, as the amount of acid is smaller for the same dose of active substance.

All acids and other excipients are generally suitable, but display somewhat different release characteristics at comparable active substance concentrations. Examples C4, C5, C7, C8, C10 and C14 have proved particularly suitable for the purposes of the invention.

Table 5 shows the in vitro releases and the characteristics of the AUC and maximum release of Examples C18 to C31 according to the invention (active substance: pimobendane; cf. Table 12) compared with reference forms C16 to C17. A 0.005 M citrate buffer adjusted to pH 5.0 was used as the test medium.

TABLE 5

| | | | | | | | | AUC 1 | |
|---|---|---|---|---|---|---|---|---|---|
| time (mins) | 0 | 2 | 4 | 6 | 10 | 15 | Max | to 15 | pH* |
| Example C16 | 0 | 11 | 11 | 15 | 15 | 14 | 16 | 194 | 5.06 |
| Example C17 | 0 | 15 | 22 | 25 | 26 | 19 | 26 | 312 | 4.85 |
| Example C18 | 0 | 53 | 61 | 74 | 87 | 91 | 91 | 1072 | 3.05 |
| Example C19 | 0 | 35 | 52 | 59 | 68 | 71 | 71 | 834 | 3.2 |
| Example C20 | 0 | 26 | 32 | 33 | 42 | 48 | 48 | 523 | 3.55 |
| Example C21 | 0 | 31 | 51 | 57 | 62 | 66 | 66 | 781 | 3.5 |
| Example C22 | 0 | 10 | 15 | 19 | 22 | 24 | 24 | 266 | 4.1 |
| Example C23 | 0 | 6 | 10 | 13 | 15 | 16 | 16 | 179 | 4.2 |
| Example C24 | 0 | 38 | 46 | 54 | 62 | 80 | 80 | 808 | 2.8 |
| Example C25 | 0 | 13 | 18 | 26 | 44 | 59 | 59 | 485 | 3.55 |
| Example C26 | 0 | 10 | 21 | 25 | 30 | 38 | 38 | 366 | 3.7 |
| Example C27 | 0 | 52 | 73 | 79 | 85 | 85 | 86 | 1079 | 2.85 |
| Example C28 | 0 | 19 | 47 | 56 | 64 | 71 | 71 | 765 | 3.3 |
| Example C29 | 0 | 7 | 19 | 22 | 25 | 26 | 26 | 298 | 3.5 |
| Example C30 | 0 | 50 | 70 | 78 | 84 | 87 | 87 | 1071 | 2.95 |
| Example C31 | 0 | 27 | 42 | 47 | 52 | 54 | 54 | 648 | 3.6 |

*This value represents the pH at the end of the release.

Interpretation of the Results:

All the Examples according to the invention are clearly superior to the reference formulation. As the content of active substance increases the in vitro release declines somewhat, as the amount of acid is smaller for the same dose of active substance.

All acids and other excipients are generally suitable, but display somewhat different release characteristics at comparable active substance concentrations. Examples C18, C19, C21, C24, C27, C28, C30 and C31 have proved particularly suitable for the purposes of the invention.

Table 6 shows the in vitro releases and the characteristics of the AUC and maximum release of Examples C35 to C52 according to the invention (active substance: lefradafiban; cf. Table 13) compared with reference forms C32 to C34. A 0.005 M citrate buffer adjusted to pH 5.0 was used as the test medium.

TABLE 6

| | | | | | | | | AUC 1 | |
|---|---|---|---|---|---|---|---|---|---|
| time (mins) | 0 | 2 | 4 | 6 | 10 | 15 | Max | to 15 | pH* |
| Example C32 | 0 | 13 | 18 | 15 | 19 | 13 | 21 | 225 | 4.95 |
| Example C33 | 0 | 10 | 13 | 18 | 17 | 18 | 20 | 218 | 4.90 |
| Example C34 | 0 | 5 | 6 | 9 | 10 | 10 | 11 | 119 | 4.95 |
| Example C35 | 0 | 102 | 106 | 101 | 100 | 101 | 106 | 1418 | 2.85 |
| Example C36 | 0 | 49 | 75 | 86 | 95 | 94 | 95 | 1166 | 3.35 |
| Example C37 | 0 | 8 | 20 | 33 | 33 | 37 | 37 | 395 | 4.00 |
| Example C38 | 0 | 62 | 77 | 78 | 79 | 83 | 83 | 1076 | 3.50 |
| Example C39 | 0 | 16 | 34 | 41 | 41 | 38 | 42 | 498 | 4.00 |
| Example C40 | 0 | 7 | 10 | 15 | 23 | 24 | 24 | 245 | 4.30 |
| Example C41 | 0 | 61 | 94 | 98 | 100 | 105 | 105 | 1318 | 2.70 |
| Example C42 | 0 | 11 | 26 | 33 | 60 | 74 | 74 | 625 | 3.50 |
| Example C43 | 0 | 7 | 10 | 16 | 29 | 44 | 44 | 322 | 3.90 |
| Example C44 | 0 | 97 | 102 | 99 | 100 | 95 | 102 | 1387 | 2.60 |
| Example C45 | 0 | 6 | 39 | 66 | 79 | 87 | 87 | 862 | 3.40 |
| Example C46 | 0 | 4 | 20 | 27 | 33 | 34 | 34 | 367 | 3.95 |
| Example C47 | 0 | 98 | 98 | 101 | 95 | 99 | 102 | 1372 | 2.75 |
| Example C48 | 0 | 40 | 62 | 69 | 69 | 72 | 72 | 904 | 3.60 |
| Example C49 | 0 | 4 | 13 | 27 | 32 | 32 | 32 | 341 | 3.95 |
| Example C50 | 0 | 95 | 100 | 101 | 105 | 104 | 106 | 1426 | 2.85 |
| Example C51 | 0 | 65 | 59 | 66 | 65 | 67 | 68 | 909 | 3.70 |
| Example C52 | 0 | 36 | 37 | 39 | 41 | 41 | 42 | 549 | 4.05 |

*This value represents the pH at the end of the release.

Interpretation of the Results:

All the Examples according to the invention are clearly superior to the reference formulation. As the content of active substance increases the in vitro release declines somewhat, as the amount of acid is smaller for the same dose of active substance.

All acids and other excipients are generally suitable, but display somewhat different release characteristics at comparable active substance concentrations. Examples C35, C36, C38, C41, C42, C44, C45, C47, C48, C50, C51 and C52 have proved particularly suitable for the purposes of the invention.

Table 7 shows the in vitro releases and the characteristics of the AUC and maximum release of Examples C54 to C59 according to the invention (active substance: Amelubant; cf. Table 14) compared with reference form C53. A mixture of water/ethanol was used as the test medium because of the very low solubility of the active substance.

TABLE 7

| | | | | | | | | | AUC 1 | |
|---|---|---|---|---|---|---|---|---|---|---|
| Time [Min] | 0 | 5 | 10 | 15 | 20 | 25 | 30 | Max | to 30 | pH* |
| Example C53 | 0 | 1 | 1 | 1 | 2 | 1 | 3 | 3 | 40 | 7.46 |
| Example C54 | 0 | 53 | 54 | 55 | 58 | 57 | 62 | 62 | 1546 | 2.46 |
| Example C55 | 0 | 34 | 39 | 39 | 41 | 43 | 44 | 44 | 1086 | 2.63 |
| Example C56 | 0 | 12 | 12 | 12 | 12 | 12 | 11 | 13 | 335 | 3.47 |
| Example C57 | 0 | 31 | 49 | 57 | 60 | 68 | 64 | 68 | 1484 | 2.46 |

*This value represents the pH at the end of the release.

Interpretation of the Results:

All the Examples according to the invention are clearly superior to the reference formulation, while in this case succinic acid starter pellets fare worse than tartaric and fumaric acid.

Examples C54 to C57 have proved particularly suitable for the purposes of the invention.

Table 8 shows the in vitro releases and the characteristics of the AUC and maximum release of Examples C60 to C61 according to the invention (active substance: telmisartan; cf. Table 15) compared with reference forms C58 and C59. A 0.0005 M citrate buffer adjusted to pH 5.0 was used as the test medium.

TABLE 8

| Time [min] | 0 | 2 | 4 | 6 | 10 | 15 | Max | AUC to 30 | pH* |
|---|---|---|---|---|---|---|---|---|---|
| Example C58 | 0 | 0 | 1 | 2 | 2 | 2 | 2 | 20.64 | 5 |
| Example C59 | 0 | 0 | 0 | 0 | 0 | 0 | 0 | 4.57 | 5 |
| Example C60 | 0 | 15 | 20 | 25 | 26 | 24 | 26 | 322.11 | 2 |
| Example C61 | 0 | 5 | 6 | 7 | 7 | 8 | 8 | 93.11 | 2 |

*This value represents the pH at the end of the release.

Interpretation of the Results:

All the Examples according to the invention are clearly superior to the reference formulation.

As the content of active substance increases the in vitro release declines somewhat, as the amount of acid is smaller for the same dose of active substance.

Examples C60 and C61 have proved particularly suitable for the purposes of the invention.

The Examples that follow are intended to illustrate the invention:

### EXAMPLES

### Example 1

TABLE 9

| | percentage composition | | | | |
|---|---|---|---|---|---|
| | | | active | | per |
| | core | insulating | substance | | capsule |
| | material | layer | layer | total | [mg] |
| tartaric acid | 61.3 | — | — | 61.3 | 176.7 |
| gum arabic | 3.1 | 2.8 | | 5.9 | 17.0 |
| talc | — | 5.6 | 3.2 | 8.8 | 25.4 |
| hydroxypropylcellulose | — | — | 4.0 | 4.0 | 11.5 |
| active substance (ethyl 3-[(2- | — | — | 20.0 | 20.0 | 57.7* |
| {[4-(hexyloxycarbonylamino- | | | | | |
| imino-methyl)-phenylamino]- | | | | | |
| methyl}-1-methyl-1H- | | | | | |
| benzimidazole-5-carbonyl)- | | | | | |
| pyridin-2-yl-amino]- | | | | | |
| propionate mesylate) | | | | | |
| total | | | | 100.0 | 288.3 |

*corresponds to 50 mg of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate (active substance base)

a) Preparation of Core Material Containing Tartaric Acid

Composition:

| | | |
|---|---|---|
| gum arabic | 1 | part by weight |
| tartaric acid | 20 | parts by weight |

1 part by weight of gum arabic is dissolved with stirring in 4 parts by weight of purified water at 50° C. 5 parts by weight of tartaric acid are then dissolved in this solution with stirring.

8.3 parts by weight of tartaric acid crystals with an average particle size of 0.4 to 0.6 mm are placed in a suitable coating apparatus fitted with an air inlet and exhaust and the container is set rotating. At an air inlet temperature of 60°-80° C. the tartaric acid crystals are sprayed with the solution of tartaric acid-gum arabic in intermittent operation and sprinkled with a total of 6.7 parts by weight of powdered tartaric acid, so as to produce roughly spherical particles.

The spherical tartaric acid core material is then dried in the rotating container at an air inlet temperature of 60°-80° C.

The core material is fractionated using a tumbler screening machine with perforated plates having nominal mesh sizes of 0.6 and 0.8 mm. The product fraction of between 0.6 and 0.8 mm is used in subsequent processing.

b) Isolation of the Core Material Containing Tartaric Acid

Composition:

| | | |
|---|---|---|
| core material containing tartaric acid | 23 | parts by weight |
| gum arabic | 1 | part by weight |
| talc | 2 | parts by weight |

1 part by weight of gum arabic is dissolved with stirring in a mixture of 6.7 parts by weight of 96% ethanol and 13.5 parts by weight of purified water. Then 2 parts by weight of talc are dispersed in the solution with stirring.

In a fluidised bed processing plant, 23 parts by weight of tartaric acid-containing core material are sprayed with the gum arabic/talc dispersion at an air entry temperature of 35°-40° C. by the under-bed spraying method.

The isolated tartaric acid-containing core material is then dried in the circulating air dryer at 40° C. for 8 hours.

To remove lumps the dried isolated tartaric acid-containing core material is screened through a screen with a nominal mesh size of 1.0 mm. The fraction of material (particle size less than 1 mm) is further processed.

c) Preparation of the Active Substance Layer

Composition:

| | | |
|---|---|---|
| isolated tartaric acid-containing core material | 91 | parts by weight |
| hydroxypropylcellulose | 5 | parts by weight |
| talc | 4 | parts by weight |
| active substance (mesylate of BIBR 1048) | 25 | parts by weight |

Hydroxypropylcellulose is dissolved in 168 parts by weight of 2-propanol with stirring and then the active substance and talc are dispersed in this solution with stirring.

In a fluidised bed processing plant, 91 parts by weight of tartaric acid-containing core material are sprayed with the dispersion containing the active substance at an air entry temperature of 20°-30° C. by the under-bed spraying method.

The pellets containing the active substance are then dried in the circulating air dryer at 35° C. for 8 hours.

To remove lumps the pellets containing the active substance are screened through a screen with a nominal mesh size of 1.25 mm. The product fraction (particle size less than 1.25 mm) is further processed.

d) Packing Into Capsules

A quantity of pellets containing active substance corresponding to 50 mg of active substance base is packed into size 1 hard gelatine capsules using a capsule filling machine.

### Example 2

TABLE 10

| | percentage composition | | | | |
|---|---|---|---|---|---|
| | | | active | | per |
| | core | insulating | substance | | capsule |
| material | layer | layer | total | [mg] | |
| tartaric acid | 38.5 | — | — | 38.5 | 55.5 |
| gum arabic | 1.9 | 1.7 | | 3.6 | 5.2 |
| talc | — | 3.5 | 6.4 | 9.9 | 14.3 |
| hydroxypropylcellulose | — | — | 8.0 | 8.0 | 11.5 |
| active substance (ethyl 3-[(2- | — | — | 40.0 | 40.0 | 57.7* |
| {[4-(hexyloxycarbonylamino- | | | | | |
| imino-methyl)-phenylamino]- | | | | | |
| methyl}-1-methyl-1H- | | | | | |
| benzimidazole-5-carbonyl)- | | | | | |
| pyridin-2-yl-amino]- | | | | | |
| propionate mesylate) | | | | | |
| total | | | | 100.0 | 144.1 |

*corresponds to 50 mg ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate (active substance base)

a) Preparation of Core Material Containing Tartaric Acid

Composition:

| | | |
|---|---|---|
| gum arabic | 1 | part by weight |
| tartaric acid | 20 | parts by weight |

1 part by weight of gum arabic is dissolved with stirring in 4 parts by weight of purified water at 50° C. 5 parts by weight of tartaric acid are then dissolved in this solution with stirring.

8.3 parts by weight of tartaric acid crystals with an average particle size of 0.4 to 0.6 mm are placed in a suitable coating apparatus fitted with an air inlet and exhaust and the container is set rotating. At an air inlet temperature of 60°-80° C. the tartaric acid crystals are sprayed with the solution of tartaric acid-gum arabic in intermittent operation and sprinkled with a total of 6.7 parts by weight of powdered tartaric acid, so as to produce roughly spherical particles.

The spherical tartaric acid core material is then dried in the rotating container at an air inlet temperature of 60°-80° C.

The core material is fractionated using a tumbler screening machine with perforated plates having nominal mesh sizes of 0.6 and 0.8 mm. The product fraction of between 0.6 and 0.8 mm is used in subsequent processing.

b) Isolation of the Core Material Containing Tartaric Acid

Composition:

| | | |
|---|---|---|
| core material containing tartaric acid | 23 | parts by weight |
| gum arabic | 1 | part by weight |
| talc | 2 | parts by weight |

1 part by weight of gum arabic is dissolved with stirring in a mixture of 6.7 parts by weight of 96% ethanol and 13.5 parts by weight of purified water. Then 2 parts by weight of talc are dispersed in the solution with stirring.

In a fluidised bed processing plant, 23 parts by weight of tartaric acid-containing core material are sprayed with the gum arabic/talc dispersion at an air entry temperature of 35°-40° C. by the under-bed spraying method.

The isolated tartaric acid-containing core material is then dried in the circulating air dryer at 40° C. for 8 hours.

To remove lumps the dried isolated tartaric acid-containing core material is screened through a screen with a nominal mesh size of 1.0 mm. The product fraction (particle size less than 1 mm) is further processed.

c) Preparation of the Active Substance Layer

Composition:

| | | |
|---|---|---|
| isolated core material containing tartaric acid | 57 | parts by weight |
| hydroxypropylcellulose | 10 | parts by weight |
| talc | 8 | parts by weight |
| active substance (mesylate of BIBR 1048) | 50 | parts by weight |

Hydroxypropylcellulose is dissolved in 335 parts by weight of 2-propanol with stirring and then the active substance and talc are dispersed in this solution with stirring.

In a fluidised bed processing plant, 91 parts by weight of isolated tartaric acid-containing core material are sprayed with the dispersion containing the active substance at an air entry temperature of 20°-30° C. by the under-bed spraying method.

The pellets containing the active substance are then dried in the circulating air dryer at 35° C. for 8 hours.

To remove lumps the pellets containing the active substance are screened through a screen with a nominal mesh size of 1.25 mm. The product fraction (particle size less than 1.25 mm) is further processed.

d) Packing Into Capsules

A quantity of pellets containing active substance corresponding to 50 mg of active substance base is packed into size 2 hard gelatine capsules using a capsule filling machine.

### Further Examples

The preparation of the following Examples usually takes place over 5 steps:
a: preparation of acid-containing core material
b: isolation of the acid-containing core material
c: preparation of the active substance layer
d: isolation of the pellets consisting of acid-containing core
e: packing into capsules

Step b is absolutely essential if there is any incompatibility between acid and active substance layer, otherwise this step may be omitted to simplify the production method. Step d is necessary if the mechanical stability of the active substance layer is insufficient to dissolve the active substance completely.

a: Examples of the Preparation of Acid-Containing Core Material

### Example a1: Preparation of Tartaric Acid-Containing Core Material

Composition:

| | | |
|---|---|---|
| gum arabic | 1 | part by weight |
| tartaric acid | 20 | parts by weight |

1 part by weight of gum arabic is dissolved with stirring in 4 parts by weight of purified water at 50° C. 5 parts by weight of tartaric acid are then dissolved in this solution with stirring.

8.3 parts by weight of tartaric acid crystals with an average particle size of 0.4 to 0.6 mm are placed in a suitable coating apparatus fitted with an air inlet and exhaust and the container is set rotating. At an air inlet temperature of 60°-80° C. the tartaric acid crystals are sprayed with the solution of tartaric acid-gum arabic in intermittent operation and sprinkled with a total of 6.7 parts by weight of powdered tartaric acid, so as to produce roughly spherical particles.

The spherical tartaric acid core material is then dried in the rotating container at an air inlet temperature of 60°-80° C.

The core material is fractionated using a tumbler screening machine with perforated plates having nominal mesh sizes of 0.6 and 0.8 mm. The product fraction of between 0.6 and 0.8 mm is used in subsequent processing.

### Example a2: Preparation of Fumaric Acid-Containing Core Material

Composition:

| | | |
|---|---|---|
| Kollidon K25 | 3 | parts by weight |
| Avicel PH101 | 20 | parts by weight |
| fumaric acid | 77 | parts by weight |

77 parts by weight of fumaric acid, 20 parts by weight of Avicel PH 101 and 3 parts by weight of Kollidon K25 are mixed for 15 minutes in a gyrowheel mixer. Then the powder mixture is transferred into a twin-screw metering device. This mixture is introduced into a twin-screw extruder of the Werner & Pfleiderer 37/18D type (or any other suitable type of extruder) at a speed of about 1 kg/h, together with water which is added by means of a ProMinent metering pump. The water is automatically regulated so as to obtain a nominal torque of approx. 19% in the extruder. The extrusion is carried out using a die plate with bores 8 mm in diameter.

The extruded strips are rounded off to form pellets in a WyPro Sphäromat, the process taking approx. 3 minutes at approx. 850 RPM.

Drying of the pellets at 80° C. for approx. 1.5 h in the GPCG1 fluidised bed dryer.

The core material is fractionated using a tumbler screening machine with different perforated plates having nominal mesh sizes of 0.71 to 1.25 mm. The suitable fractions of materials of between 0.71 and 0.90 or 0.90 and 1.12 mm are used in subsequent processes.

### Example a3: Preparation of Succinic Acid-Containing Core Material

Composition:

| | | |
|---|---|---|
| Kollidon K25 | 3 | parts by weight |
| Avicel PH101 | 20 | parts by weight |
| succinic acid | 77 | parts by weight |

77 parts by weight succinic acid, 20 parts by weight Avicel PH 101 and 3 parts by weight Kollidon K25 are mixed for 15 minutes in a gyrowheel mixer. Then the powder mixture is transferred into a forced mixer. Water is added to this mixture, with stirring, until an extrudable homogeneous mass is formed. This mixture is then further processed analogously to prepare the starter cores containing fumaric acid.

The product fractions of between 0.71 and 0.90 or 0.90 and 1.12 mm which are suitable in each case are used in the subsequent processes.

### Example a4: Preparation of Citric Acid-Containing Core Material

Composition:

| | | |
|---|---|---|
| Kollidon K90 | 1 | part by weight |
| talc | 1 | part by weight |
| citric acid | 20 | parts by weight |

1 part by weight of Kollidon K90 is dissolved in 6 parts by weight of isopropanol with stirring and 1 part by weight of talc is suspended therein. 3 parts by weight of citric acid are dissolved in another 6 parts by weight of isopropanol, with stirring.

In a fluidised bed processing plant, 17 parts by weight of isolated citric acid-containing core material measuring 0.5-0.6 mm are sprayed with the two dispersions/solutions containing the active substance at an air entry temperature of 20°-30° C. by the under-bed spraying method, using a three-headed nozzle, so as to obtain substantially spherical particles.

The pellets containing the active substance are then dried in the circulating air dryer at 35° C. for 8 hours.

The spherical tartaric acid core material is then dried in the rotating container at an air inlet temperature of 60°-80° C.

The core material is fractionated using a tumbler screening machine with perforated plates having nominal mesh sizes of 0.6 and 0.8 mm. The product fraction between 0.6 and 0.8 mm is used for further processing.

b: Example of the Isolation of the Acid-Containing Core Material

Composition:

| | | |
|---|---|---|
| acid-containing core material | 23 | parts by weight |
| gum arabic | 1 | part by weight |
| talc | 2 | parts by weight |

1 part by weight of gum arabic is dissolved with stirring in a mixture of 6.7 parts by weight of 96% ethanol and 13.5 parts by weight of purified water. Then 2 parts by weight of talc are dispersed in the solution with stirring.

In a fluidised bed processing plant, 23 parts by weight of acid-containing core material are sprayed with the gum arabic/talc dispersion at an air entry temperature of 35°-40° C. by the under-bed spraying method.

The isolated tartaric acid-containing core material is then dried in the circulating air dryer at 40° C. for 8 hours.

To remove lumps the dried isolated tartaric acid-containing core material is screened through a screen with a nominal mesh size of 1.0 mm. The fraction of material (particle size less than 1 mm) is further processed.

c: Examples of the Preparation of the Active Substance Layer

The active substance layer is generally prepared in the same way, but with variations in the nature and quantity of the active substance, the nature and quantity of the binder, the amount of talc and isopropanol or the amount of ethanol. The preparation is therefore only described for Example C4, and the particular compositions for each active substance are shown in table form.

Preparation of Example C4:

Composition:

| | | |
|---|---|---|
| isolated tartaric acid-containing core material | 74 | parts by weight |
| hydroxypropylcellulose | 49 | parts by weight |
| talc | 12 | parts by weight |
| active substance (e.g. flibanserin) | 25 | parts by weight |

Hydroxypropylcellulose is dissolved in 492 parts by weight of 2-propanol with stirring and then the active substance and talc are dispersed in this solution with stirring.

In a fluidised bed processing plant, 74 parts by weight of isolated tartaric acid-containing core material are sprayed with the dispersion containing the active substance at an air entry temperature of 20°-30° C. by the under-bed spraying method.

The pellets containing the active substance are then dried at 35° C. in the circulating air dryer for 8 hours.

To remove lumps the pellets containing the active substance are screened through a screen with a nominal mesh size of 1.25 mm. The product fraction (particle size less than 1.25 mm) is further processed.

Table 11 shows the composition of Examples C1-C15 (active substance flibanserin). In each case parts by weight are specified which correspond to the active substance content determined experimentally, i.e. the spray losses, which may fluctuate somewhat from one batch to the next, were compensated in the calculation in each case so as to obtain truly comparable data.

Examples C1-C3 contain a commercially available neutral core instead of the acid-containing starter cores according to the invention. These cores serve as reference values for the in vitro testing (see above).

Table 12 shows the composition of Examples C16-C31 (active substance pimobendane). In each case parts by weight are specified which correspond to the active substance content determined experimentally, i.e. the spray losses, which may fluctuate somewhat from one batch to the next, were compensated in the calculation in each case so as to obtain truly comparable data.

Examples C16-C17 contain a commercially available neutral core instead of the acid-containing starter cores according to the invention. These cores serve as reference values for the in vitro testing (see above).

Table 13 shows the composition of Examples C32-C52 (active substance lefradafiban, BIBU 104). In each case parts by weight are specified which correspond to the active substance content determined experimentally, i.e. the spray losses, which may fluctuate somewhat from one batch to the next, were compensated in the calculation in each case so as to obtain truly comparable data.

Examples C32-C34 contain a commercially available neutral core instead of the acid-containing starter cores according to the invention. These cores serve as reference values for the in vitro testing (see above).

Table 14 shows the composition of Examples C53-C57 (active substance BIIL 284=amelubant). In each case parts by weight are specified which correspond to the active substance content determined experimentally, i.e. the spray losses, which may fluctuate somewhat from one batch to the next, were compensated in the calculation in each case so as to obtain truly comparable data.

Example C53 contains a commercially available neutral core instead of the acid-containing starter cores according to the invention. This core serves as a reference value for the in vitro testing (see above).

Table 15 shows the composition of Examples C58-C61 (active substance pimobendane). In each case parts by weight are specified which correspond to the active substance content determined experimentally, i.e. the spray losses, which may fluctuate somewhat from one batch to the next, were compensated in the calculation in each case so as to obtain truly comparable data.

Examples C58-C59 contain a commercially available neutral core instead of the acid-containing starter cores according to the invention. These cores serve as reference values for the in vitro testing (see above).

TABLE 11

| | | | succinic acid | fumaric | | | | | isopropanol | active |
|---|---|---|---|---|---|---|---|---|---|---|
| | neutral | tartaric acid | acid | acid | fliban- | povidone | | | (only for | substance |
| | pellets | pellets | pellets | pellets | serine | K25 | talc | Klucel EF | preparation) | content [%] |
| Example C1 | 200 | | | | 17 | | 8 | 4 | 268 | 6.9 |
| Example C2 | 200 | | | | 76 | | 38 | 19 | 885 | 22.7 |
| Example C3 | 200 | | | | 127 | | 64 | 32 | 1272 | 31.4 |
| Example C4 | | 200 | | | 35 | | 17 | 9 | 348 | 12.6 |
| Example C5 | | 200 | | | 89 | | 44 | 22 | 888 | 24.8 |
| Example C6 | | 200 | | | 204 | | 102 | 51 | 2040 | 37.7 |
| Example C7 | | | | 200 | 43 | | 22 | 11 | 432 | 14.9 |
| Example C8 | | | | 200 | 96 | | 48 | 24 | 960 | 24.7 |
| Example C9 | | | | 200 | 170 | | 85 | 43 | 1704 | 34.6 |
| Example C10 | | | 200 | | 22 | | 11 | 5 | 216 | 9.2 |
| Example C11 | | | 200 | | 84 | | 42 | 21 | 840 | 22.7 |
| Example C12 | | | 200 | | 168 | | 84 | 42 | 1680 | 36.5 |
| Example C13 | | | 200 | | 31 | 4 | 16 | | 488 | 11.9 |
| Example C14 | | | 200 | | 84 | 11 | 42 | | 999 | 24.4 |
| Example C15 | | | 200 | | 156 | 20 | 78 | | 1454 | 35.5 |

TABLE 12

| | | tartaric | succinic | fumaric | | | | | isopropanol | active |
|---|---|---|---|---|---|---|---|---|---|---|
| | neutral | acid | acid | acid | pimo- | | | | (only for | substance |
| | pellets | starter | starter | starter | bendane | povidone K90 | talc | Klucel EF | preparation) | content [%] |
| Example C16 | 200 | | | | 10 | 3 | 5 | | 14 | 5 |
| Example C17 | 200 | | | | 28 | 9 | 14 | | 38 | 11 |
| Example C18 | | 200 | | | 12 | 4 | 6 | | 16 | 5 |
| Example C19 | | 200 | | | 30 | 10 | 15 | | 40 | 11 |
| Example C20 | | 200 | | | 55 | 18 | 27 | | 73 | 18 |
| Example C21 | | | 200 | | 13 | 4 | 6 | | 17 | 5 |
| Example C22 | | | 200 | | 41 | 14 | 20 | | 54 | 14 |
| Example C23 | | | 200 | | 62 | 21 | 31 | | 83 | 21 |
| Example C24 | | | | 200 | 16 | 5 | 8 | | 21 | 7 |
| Example C25 | | | | 200 | 30 | 10 | 15 | | 40 | 12 |
| Example C26 | | | | 200 | 81 | 27 | 41 | | 108 | 24 |
| Example C27 | | 200 | | | 10 | | 5 | 3 | 13 | 4 |
| Example C28 | | 200 | | | 33 | | 17 | 11 | 46 | 12 |
| Example C29 | | 200 | | | 45 | | 23 | 15 | 60 | 16 |
| Example C30 | | 200 | | | 13 | 2 | 6 | 2 | 17 | 5 |
| Example C31 | | 200 | | | 37 | 6 | 19 | 6 | 50 | 13 |

TABLE 13

| | | citric | tartaric | succinic | fumaric | | | | | isopropanol | exp. active |
|---|---|---|---|---|---|---|---|---|---|---|---|
| | neutral | acid | acid | acid | acid | | povidone | | | (only for | substance |
| | pellets | starter | starter | starter | starter | lefradafiban | K90 | talc | Klucel EF | preparation) | content [%] |
| Example C32 | 200 | | | | | 28 | 9 | 14 | | 184 | 10.8 |
| Example C33 | 200 | | | | | 94 | 31 | 47 | | 624 | 25.3 |
| Example C34 | 200 | | | | | 163 | 54 | 82 | | 1088 | 33.3 |
| Example C35 | | | 200 | | | 28 | 9 | 14 | | 184 | 10.9 |
| Example C36 | | | 200 | | | 82 | 27 | 41 | | 544 | 23.1 |
| Example C37 | | | 200 | | | 160 | 53 | 80 | | 1064 | 33.5 |
| Example C38 | | | | 200 | | 24 | 8 | 12 | | 160 | 10.1 |
| Example C39 | | | | 200 | | 82 | 27 | 41 | | 544 | 23.3 |
| Example C40 | | | | 200 | | 163 | 54 | 82 | | 1088 | 32.7 |
| Example C41 | | | | | 200 | 24 | 8 | 12 | | 160 | 9.8 |
| Example C42 | | | | | 200 | 86 | 29 | 43 | | 576 | 23.6 |
| Example C43 | | | | | 200 | 94 | 31 | 47 | | 624 | 32.1 |
| Example C44 | | | 200 | | | 22 | | 11 | 7 | 144 | 9.0 |
| Bespiel C45 | | | 200 | | | 82 | | 41 | 27 | 544 | 22.8 |
| Example C46 | | | 200 | | | 175 | | 88 | 58 | 1168 | 33.5 |
| Example C47 | | | 200 | | | 24 | 4 | 12 | 4 | 160 | 10.5 |
| Example C48 | | | 200 | | | 94 | 16 | 47 | 16 | 624 | 24.8 |
| Example C49 | | | 200 | | | 192 | 32 | 96 | 32 | 1280 | 34.7 |
| Example C50 | | 200 | | | | 24 | 8 | 12 | | 160 | 9.8 |
| Example C51 | | 200 | | | | 82 | 27 | 41 | | 544 | 23.4 |
| Example C52 | | 200 | | | | 175 | 58 | 88 | | 1168 | 34.4 |

TABLE 14

| | | citric | tartaric | succinic | fumaric | | | | | isopropanol | exp. active |
|---|---|---|---|---|---|---|---|---|---|---|---|
| | neutral | acid | acid | acid | acid | | Povidone | | | (only for | substance |
| | pellets | starter | starter | starter | starter | amelubant | K90 | talc | Klucel EF | preparation) | content |
| Example C53 | 200 | | | | | 13 | 3 | 3 | | 104 | 6 |
| Example C54 | | | 200 | | | 23 | 5 | 5 | | 184 | 10 |
| Example C55 | | | 200 | | | 48 | 10 | 10 | | 384 | 18 |
| Example C56 | | | | 200 | | 38 | 8 | 8 | | 304 | 15 |
| Example C57 | | | | | 200 | 33 | 7 | 7 | | 264 | 13 |

TABLE 15

| | | citric | tartaric | succinic | fumaric | | | | | isopropanol | exp. active |
|---|---|---|---|---|---|---|---|---|---|---|---|
| | neutral | acid | acid | acid | acid | | Povidone | | | (only for | substance |
| | pellets | starter | starter | starter | starter | telmisartan | K90 | talc | Klucel EF | preparation) | content |
| Example C58 | 200 | | | | | 7 18 | 2 | 3 9 | | 85 | 3 |
| Example C59 | 200 | | | | | 7 | 4 | 3 | | 220 | 8 |
| Example C60 | | | 200 | | | 20 | 2 | 10 | | 85 | 3 |
| Example C61 | | | 200 | | | | 5 | | | 250 | 9 |

d: Example of the Isolation of the Pellets Containing Active Substance

Composition:

| | | |
|---|---|---|
| pellets containing active substance | 23 | parts by weight |
| gum arabic | 1 | part by weight |
| talc | 2 | parts by weight |

1 part by weight of gum arabic is dissolved with stirring in a mixture of 6.7 parts by weight of 96% ethanol and 13.5 parts by weight of purified water. Then 2 parts by weight of talc are dispersed in the solution with stirring.

In a fluidised bed processing plant, 23 parts by weight of active substance-containing core material are sprayed with the gum arabic/talc dispersion at an air entry temperature of 35°-40° C. by the under-bed spraying method.

The isolated tartaric acid-containing core material is then dried in the circulating air dryer at 40° C. for 8 hours.

To remove lumps the dried active substance-containing pellets are screened through a screen with a nominal mesh size of 1.25 mm. The product fraction (particle size less than 1.25 mm) is further processed.

e) Packing Into Capsules

A quantity of pellets containing active substance corresponding to the desired dosage in each case is packed into hard gelatine capsules of suitable size using a capsule filling machine.

1. Pharmaceutical composition with a bioavailability of the active substance which is substantially independent of the gastric pH, for oral administration of active substances with pH-dependent solubilities and a dose number of more than 1 at a pH>5, comprising a plurality of pellets synthesised in each case from
a) a core material,
b) an optional insulating layer,
c) an active substance layer and
d) an optional coating,
wherein the core material consists of one or more pharmaceutically acceptable organic acid(s) with a water solubility of more than 1 g/250 ml at 20° C., optionally with the addition of binders or other technological adjuvants.

2. Pharmaceutical composition according to claim 1, wherein the pharmaceutically acceptable organic acid is chosen from tartaric acid, fumaric acid, succinic acid, citric acid, malic acid, glutamic acid and aspartic acid or one of the hydrates or acid salts thereof.

3. Pharmaceutical composition according to claim 2, characterised in that the pharmaceutically acceptable organic acid is chosen from tartaric acid, fumaric acid, citric acid or succinic acid.

4. Pharmaceutical composition according to claim 2, characterised in that the pharmaceutically acceptable organic acid is tartaric acid.

5. Pharmaceutical composition according to claim 1, wherein the binder is selected from the group comprising the hydroxypropylcelluloses, the hydroxypropylmethylcelluloses, the methylcelluloses, the hydroxyethylcelluloses, the carboxymethylcelluloses, the polyvinylpyrrolidones, the copolymers of N-vinylpyrrolidone and vinyl acetate or combinations of these polymers.

6. Pharmaceutical composition according to claim 1 wherein the core material has an average particle size of 0.4 to 1.5 mm.

7. Pharmaceutical composition according to claim 1, wherein the insulating layer consists of a water-soluble polymer, optionally with the addition of suitable plasticisers, separating agents and pigments.

8. Pharmaceutical composition according to claim 7, wherein said water-soluble polymer consists of gum arabic or a partially or totally synthetic polymer selected from among the hydroxypropylcelluloses, the hydroxypropylmethylcelluloses, the methylcelluloses, the hydroxyethylcelluloses, the carboxymethylcelluloses, the polyvinylpyrrolidones, the copolymers of N-vinylpyrrolidone and vinyl acetate or combinations of said polymers.

9. Pharmaceutical composition according to claim 1, wherein the coating consists of film-forming agents, plasticisers and optionally pigments.

10. Pharmaceutical composition according to claim 1 wherein said pellets containing active substance are packed into hard capsules.

11. Process for preparing a pharmaceutical composition for oral administration containing an active substance with pH-dependent solubility characteristics and a dose number>>1 at pH>5 or one of the physiologically acceptable salts thereof, comprising the steps of:
a) synthesising the core material from one or more pharmaceutically acceptable organic acid(s) with a water solubility of more than 1 g/250 ml at 20° C., optionally with the addition of binders or other technological adjuvants, by pan methods, on pelleting plates or by extrusion/spheronisation,
b) applying an insulating layer consisting of one or more water-soluble, pharmaceutically acceptable polymers, optionally with the addition of plasticisers, separating agents and/or pigments, to the core material,
c) applying the active substance from a dispersion containing binder and optionally separating agent, and simultaneously or subsequently drying to eliminate the dispersing agent,
d) optionally applying a coating of film-forming agents, plasticisers and optionally pigments and
e) packing the pellets containing active substance thus obtained into hard capsules.
